SEQUENCE LISTING (10 sequences)

SEQ ID NO: 1 - protein, 30 aa, Homo sapiens
HAEGTFTSDVSSYLEGQAAKEFIAWLVKGR

SEQ ID NO: 2 - protein, 30 aa, Artificial Sequence
  Synthetic analogue of glucagon-like peptide-1 (GLP-1)
HXEGTFTSDVSSYLEGQAAKEFIAWLVKXR

SEQ ID NO: 3 - protein, 30 aa, Artificial Sequence; identical to SEQ ID NO: 2
  Synthetic analogue of glucagon-like peptide-1 (GLP-1)

SEQ ID NO: 4 - protein, 29 aa, Artificial Sequence
  Synthetic analogue of glucagon-like peptide-1 (GLP-1)
XEGTFTSDVSSYLEGQAAKEFIAWLVKXR

SEQ ID NO: 5 - protein, 30 aa, Artificial Sequence; identical to SEQ ID NO: 2
  Synthetic analogue of glucagon-like peptide-1 (GLP-1)

SEQ ID NO: 6 - protein, 29 aa, Artificial Sequence
  Synthetic analogue of glucagon-like peptide-1 (GLP-1)
XEGTFTSDVSSYLEGQAAKEFIAWLVKXR

SEQ ID NO: 7 - protein, 30 aa, Artificial Sequence; identical to SEQ ID NO: 2
  Synthetic analogue of glucagon-like peptide-1 (GLP-1)

SEQ ID NO: 8 - protein, 28 aa, Artificial Sequence
  Synthetic analogue of glucagon-like peptide-1 (GLP-1)
XEGTFTSDVSSYLEGQAAKEFIAWLVKX

SEQ ID NO: 9 - protein, 29 aa, Artificial Sequence
  Synthetic analogue of glucagon-like peptide-1 (GLP-1)
HXEGTFTSDVSSYLEGQAAKEFIAWLVKX

SEQ ID NO: 10 - protein, 30 aa, Artificial Sequence; identical to SEQ ID NO: 2
  Synthetic analogue of glucagon-like peptide-1 (GLP-1)

(19) United States
(12) Patent Application Publication
(10) Pub. No.: US 2013/0030148 A1
(43) Pub. Date: Jan. 31, 2013

(54) PROCESS FOR THE SYNTHESIS OF (AIB8,35)HGLP-1(7-36)-NH2

(71) Applicants: Zheng Xin Dong, Holliston, MA (US); Thomas Ciaran Loughman, Dublin (IE); Fionn Hurley, Dublin (IE); Steven R. Johnson, Monmouth Junction, NJ (US)

(72) Inventors: Zheng Xin Dong, Holliston, MA (US); Thomas Ciaran Loughman, Dublin (IE); Fionn Hurley, Dublin (IE); Steven R. Johnson, Monmouth Junction, NJ (US)

(21) Appl. No.: 13/120,195

(22) PCT Filed: Sep. 22, 2009
(86) PCT No.: PCT/US09/05265

§ 371 Date: May 16, 2012

Related U.S. Application Data
(60) Provisional application No. 61/192,939, filed on Sep. 22, 2008.

(51) Int. Cl. C07K 1/04

(52) U.S. Cl. 530/308

(57) ABSTRACT

The present invention relates to a process for the large-scale synthesis of (Aib^{8,35})hGLP-1(7-36)-NH2 (SEQ ED NO:2), i.e., His-Aib-Glu-Gly-Thr-Phe-Thr-Ser-Asp-Val-Ser-Ser-Tyr-Leu-Glu-Gly-Gb-Ala-Ala-Lys-Glu-Phe-Ile-Ala-Trp-Leu-Val-Lys-Aib-Arg-NH2 (SEQ ID NO:2), which comprises solid-phase Fmoc-chemistry.

### FIELD OF THE INVENTION

The present invention relates to a novel process for the large-scale synthesis of (Aib^{8,35})hGLP-1(7-36)-NH₂ (SEQ ID NO:2), i.e., His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg-NH₂ (SEQ ID NO:2), which comprises solid-phase Fmoc-chemistry.

### BACKGROUND ART

Glucagon-like peptide-1 (7-36) amide (GLP-1) (SEQ ID NO:1) is synthesized in the intestinal L-cells by tissue-specific post-translational processing of the glucagon precursor preproglucagon and is released into the circulation in response to a meal. The therapeutic potential of GLP-1 was suggested following the observation that a single subcutaneous dose of GLP-1 could completely normalize postprandial glucose levels in patients with non-insulin-dependent diabetes mellitus (NIDDM) (Gutniak, M. K., et al., 1994, Diabetes Care, 14:1039-44). This effect was thought to be mediated both by increased insulin release and by a reduction in glucagon secretion. GLP-1 is, however, metabolically unstable, having a plasma half-life of only 1-2 minutes in vivo. Exogenously administered GLP-1 is also rapidly degraded (Deacon, C. F., et al., 1995, Diabetes, 44:1126-1131).

(Aib^{8,35})hGLP-1(7-36)-NH₂ (SEQ ID NO:2) is disclosed in PCT Publication No. WO 00/34331, the content of which is incorporated herein in its entirety, as being more active and/or more metabolically stable than the native GLP-1. However, the synthetic description for (Aib^{8,35})hGLP-1(7-36)-NH₂ (SEQ ID NO:2) provided at pages 18-19 of WO 00/34331 is not suitable for commercial scale production of the peptide, because the MBHA (4-methylbenzhydrylamine) resin used therein requires the peptide be removed using hydrofluoric acid. Outside of the safety concerns of using this extremely corrosive material at large scale, special equipment would have been required to permit its use. In general, hydrofluoric acid-based cleavage schemes require significant investment to ensure they are safe and scaleable to industrial scale. As such, there is a need for developing an efficient large-scale method for producing (Aib^{8,35})hGLP-1(7-36)-NH₂ (SEQ ID NO:2).

### SUMMARY OF THE INVENTION

The present invention provides a novel process for the synthesis of (Aib^{8,35})hGLP-1(7-36)-NH₂ (SEQ ID NO:2), which comprises stepwise solid-phase Fmoc-chemistry.

In one aspect, the present invention provides a process for the synthesis of (Aib^{8,35})hGLP-1(7-36)-NH₂ (SEQ ID NO:2), comprising the steps of:

(a) successively coupling Fmoc-amino acids, from the C-terminus to the N-terminus of (Aib^{8,35})hGLP-1(8-35)-NH₂ (SEQ ID NO:8), with a sidechain-protected Arg resin, wherein the Fmoc group is removed from the N-terminus after each successive coupling step, to yield a sidechain-protected Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:4);

(b) coupling sidechain-protected Boc-His-OH with the sidechain-protected Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:4) to yield a sidechain-protected Boc-His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:5);

(c) treating the sidechain-protected Boc-His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:5) with a cleavage cocktail and removing the sidechain-protecting groups and the N-terminus protecting group therefrom to yield crude (Aib^{8,35})hGLP-1(7-36)-NH₂ (SEQ ID NO:2); and

(d) isolating and purifying the crude (Aib^{8,35})hGLP-1(7-36)-NH₂ (SEQ ID NO:2) to yield purified (Aib^{8,35})hGLP-1(7-36)-NH₂ (SEQ ID NO:2).

A preferred embodiment of the immediately foregoing aspect of the present invention further comprises the steps of:

(a-1) deprotecting an Fmoc-protected resin capable of generating a peptide amide to remove the Fmoc group from the resin;

(a-2) attaching sidechain-protected Fmoc-Arg-OH onto the resin to yield a sidechain-protected Fmoc-Arg resin; and (a-3) removing the Fmoc group from the sidechain-protected Fmoc-Arg resin to yield a sidechain-protected Arg resin;

which precede the step (a).

A preferred embodiment of the immediately foregoing aspect of the present invention is characterized in that:

said sidechain-protected Fmoc-Arg-OH in the step (a-2) is Fmoc-Arg(Pbf)-OH;

said sidechain-protected Fmoc-Arg resin is Fmoc-Arg(Pbf) resin;

said sidechain-protected Arg resin is sidechain-protected Arg(Pbf) resin;

said Fmoc-amino acids from the C-terminus to the N-terminus of the formula (Aib^{8,35})hGLP-1(8-35)-NH₂ (SEQ ID NO:8) are Fmoc-Aib-OH, Fmoc-Lys(Boc)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(Boc)-OH, Fmoc-Ala-OH, Fmoc-Ala-OH, Fmoc-Gln(Trt)-OH, Fmoc-Gly-OH, Fmoc-Glu(OtBu)-OH, Fmoc-Leu-OH, Fmoc-Tyr(tBu)-OH, Fmoc-Ser(tBu)-OH, Fmoc-Ser(tBu)-OH, Fmoc-Val-OH, Fmoc-Asp(OtBu)-OH, Fmoc-Ser(tBu)-OH, Fmoc-Thr(tBu)-OH, Fmoc-Phe-OH, Fmoc-Thr(tBu)-OH, Fmoc-Gly-OH, Fmoc-Glu(OtBu)-OH, and Fmoc-Aib-OH;

said sidechain-protected Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:4) is Aib-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Aib-Arg(Pbf) resin (SEQ ID NO:6);

said sidechain-protected Boc-His-OH is Boc-His(Trt)-OH;

said sidechain-protected Boc-His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:5) is Boc-His(Trt)-Aib-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Aib-Arg(Pbf) resin (SEQ ID NO:7); and

said cleavage cocktail is selected from the group consisting of TFA/TIPS/water cleavage cocktail, TFA/TIPS/DCM cleavage cocktail, TFA/phenol/water/TIPS cleavage cocktail, TFA/phenol/water/thioanisole/EDT cleavage cocktail, TFA/phenol/water/thioanisole/1-dodecanethiol cleavage cocktail, TFA/DTT/water/TIPS cleavage cocktail, TFA/phenol cleavage cocktail, TFA/phenol/methanesulfonic acid cleavage cocktail, TFA/thioanisole/EDT/anisole cleavage cocktail, TFA/TES cleavage cocktail, TFA/water cleavage cocktail, TFA/DCM/indole cleavage cocktail, and TFA/TIPS cleavage cocktail.

A preferred embodiment of the immediately foregoing aspect of the present invention is characterized in that said resin capable of generating a peptide is selected from the group consisting of Fmoc-Rink amide-MBHA resin, Fmoc-Rink amide-AM resin, a PEG-based Fmoc-Rink amide resin, and Sieber amide resin.

A preferred embodiment of the immediately foregoing aspect of the present invention is characterized in that:

said cleavage cocktail is selected from the group consisting of TFA/TIPS/water cleavage cocktail, TFA/TIPS/DCM cleavage cocktail, and TFA/water cocktail; and

said resin capable of generating a peptide amide is selected from the group consisting of Fmoc-Rink amide-MBHA resin, Fmoc-Rink amide-AM resin, and a PEG-based Fmoc-Rink amide resin.

A preferred embodiment of the immediately foregoing aspect of the present invention is characterized in that said resin capable of generating a peptide amide is Fmoc-Rink amide-MBHA resin.

A preferred embodiment of the immediately foregoing aspect of the present invention is characterized in that the step (d) comprises the steps of:

(d-1) filtering to remove the resin to yield a (Aib^{8,35})hGLP-1(7-36)-NH₂ (SEQ ID NO:2)/cleavage cocktail filtrate;

(d-2) concentrating the (Aib^{8,35})hGLP-1(7-36)-NH₂ (SEQ ID NO:2)/cleavage cocktail filtrate;

(d-3) precipitating crude (Aib^{8,35})hGLP-1(7-36)-NH₂ (SEQ ID NO:2) from the concentrated (Aib^{8,35})hGLP-1(7-36)-NH₂ (SEQ ID NO:2)/cleavage cocktail filtrate;

(d-4) slurrying the crude precipitated (Aib^{8,35})hGLP-1(7-36)-NH₂ (SEQ ID NO:2) in ammonium acetate buffer to perform N—O shift reversal;

(d-5) adjusting the pH of the slurry to yield a solution of (Aib^{8,35})hGLP-1(7-36)-NH₂ (SEQ ID NO:2); and

(d-6) isolating and purifying (Aib^{8,35})hGLP-1(7-36)-NH₂ (SEQ ID NO:2).

A preferred embodiment of the immediately foregoing aspect of the present invention is characterized in that said N—O shift reversal is performed by holding the crude precipitated (Aib^{8,35})hGLP-1(7-36)-NH₂ (SEQ ID NO:2) in a slightly basic medium and then bringing the pH back down to about from 3 to 3.7.

A preferred embodiment of the immediately foregoing aspect of the present invention is characterized in that said removal of the Fmoc group from the resin is performed using piperidine in DMF.

A preferred embodiment of the immediately foregoing aspect of the present invention is characterized in that the concentration of said piperidine in DMF is about 25% (v/v).

A preferred embodiment of the immediately foregoing aspect of the present invention is characterized in that the amino acid residues of (Aib^{8,35})hGLP-1(7-36)-NH₂ (SEQ ID NO:2) are coupled using a coupling reagents combination selected from the group consisting of TBTU/HOBt, TBTU/HBTU/DIEA, HATU/DIEA, HCTU/DIEA, TBTU/HOBt/DIEA, DIC/HOBt, DIC/HOAt, HATU/HOBt/DIEA, and HCTU/HOBt/DIEA.

A preferred embodiment of the immediately foregoing aspect of the present invention is characterized in that:

the first 29 amino acid residues of (Aib^{8,35})hGLP-1(7-36)-NH₂ (SEQ ID NO:2) from the C-terminus are coupled using a coupling reagents combination of either TBTU/HOBt or TBTU/HBTU/DIEA; and

the N-terminal histidine is coupled using a coupling reagents combination selected from the group consisting of HATU/DIEA, HCTU/DIEA, TBTU/HBTU/DIEA, TBTU/HOBt/DIEA, DIC/HOBt, DIC/HOAt, HATU/HOBt/DIEA, and HCTU/HOBt/DIEA.

A preferred embodiment of the immediately foregoing aspect of the present invention is characterized in that:

said coupling reagents combination used for coupling the first 29 amino acid residues of (Aib⁸³⁵)hGLP-1(7-36)-NH₂ (SEQ ID NO:2) from the C-terminus is TBTU/HOBt; and

said coupling reagents combination used for coupling the N-terminal histidine is HATU/DIEA.

A preferred embodiment of the immediately foregoing aspect of the present invention is characterized in that:

the first 29 amino acid residues of (Aib^{8,35})hGLP-1(7-36)-NH₂ (SEQ ID NO:2) from the C-terminus are coupled using about 3.0 equivalents of each Fmoc-amino acid, about 2.94 equivalents of TBTU, about 2.94 equivalents of HOBt, and about 4.5 equivalents of DIEA, in about 5 volumetric excesses of DMF; and

the N-terminal histidine is coupled using about 3.4 equivalents of Boc-His(Trt)-OH, about 4.08 equivalents of HATU, and about 9.0 equivalents of DIEA, in about 5 volumetric excesses of DMF.

In another aspect, the present invention provides a process for the synthesis of (Aib^{8,35})hGLP-1(7-36)-NH₂ (SEQ ID NO:2) according to claim 1, comprising the steps of:

(a) successively coupling Fmoc-amino acids, from the C-terminus to the N-terminus of (Aib^{8,35})hGLP-1(7-35)-NH₂ (SEQ ID NO:9), with a sidechain-protected Arg resin, wherein the Fmoc group is removed from the N-terminus after each successive coupling step, to yield a sidechain-protected His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:3);

(b) treating the sidechain-protected His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:3) with a cleavage cocktail and removing sidechain-protecting groups therefrom to yield crude (Aib^{8,35})hGLP-1(7-36)-NH₂ (SEQ ID NO:2); and

(c) isolating and purifying the crude (Aib^{8,35})hGLP-1(7-36)-NH₂ (SEQ ID NO:2) to yield purified (Aib^{8,35})hGLP-1(7-36)-NH₂ (SEQ ID NO:2).

A preferred embodiment of the immediately foregoing aspect of the present invention further comprises the steps of:

(a-1) deprotecting an Fmoc-protected resin capable of generating a peptide amide to remove the Fmoc group from the resin;

(a-2) attaching sidechain-protected Fmoc-Arg-OH onto the resin to yield a sidechain-protected Fmoc-Arg resin; and

(a-3) removing the Fmoc group from the sidechain-protected Fmoc-Arg resin to yield a sidechain-protected Arg resin;

which precede the step (a).

A preferred embodiment of the immediately foregoing aspect of the present invention is characterized in that:

said sidechain-protected Fmoc-Arg-OH in the step (a-2) is Fmoc-Arg(Pbf)-OH;

said sidechain-protected Fmoc-Arg resin is Fmoc-Arg(Pbf)-OH and Fmoc-Arg(Pbf) resin;

said sidechain-protected Arg resin is sidechain-protected Arg(Pbf) resin;

said Fmoc-amino acids from the C-terminus to the N-terminus of the formula (Aib^{8,35})hGLP-1(7-35)-NH₂ (SEQ ID NO:9) are Fmoc-Aib-OH, Fmoc-Lys(Boc)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(Boc)-OH, Fmoc-Ala-OH, Fmoc-Ala-OH, Fmoc-Gln(Trt)-OH, Fmoc-Gly-OH, Fmoc-Glu(OtBu)-OH, Fmoc-Leu-OH, Fmoc-Tyr(tBu)-OH, Fmoc-Ser(tBu)-OH, Fmoc-Ser(tBu)-OH, Fmoc-Val-OH, Fmoc-Asp(OtBu)-OH, Fmoc-Ser(tBu)-OH, Fmoc-Thr(tBu)-OH, Fmoc-Phe-OH, Fmoc-Thr(tBu)-OH, Fmoc-Gly-OH, Fmoc-Glu(OtBu)-OH, Fmoc-Aib-OH, and Fmoc-His(Trt)-OH;

said sidechain-protected His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:3) is His(Trt)-Aib-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Aib-Arg(Pbf) resin (SEQ ID NO:10); and

said cleavage cocktail is selected from the group consisting of TFA/TIPS/water cleavage cocktail, TFA/TIPS/DCM cleavage cocktail, TFA/phenol/water/TIPS cleavage cocktail, TFA/phenol/water/thioanisole/EDT cleavage cocktail, TFA/phenol/water/thioanisole/1-dodecanethiol cleavage cocktail, TFA/DTT/water/TIPS cleavage cocktail, TFA/phenol cleavage cocktail, TFA/phenol/methanesulfonic acid cleavage cocktail, TFA/thioanisole/EDT/anisole cleavage cocktail, TFA/TES cleavage cocktail, TFA/water cleavage cocktail, TFA/DCM/indole cleavage cocktail, and TFA/TIPS cleavage cocktail.

A preferred embodiment of the immediately foregoing aspect of the present invention is characterized in that said resin capable of generating a peptide is selected from the group consisting of Fmoc-Rink amide-MBHA resin, Fmoc-Rink amide-AM resin, a PEG-based Fmoc-Rink amide resin, and Sieber amide resin.

A preferred embodiment of the immediately foregoing aspect of the present invention is characterized in that:

said cleavage cocktail is selected from the group consisting of TFA/TIPS/water cleavage cocktail, TFA/TIPS/DCM cleavage cocktail, and TFA/water cocktail; and

said resin capable of generating a peptide amide is selected from the group consisting of Fmoc-Rink amide-MBHA resin, Fmoc-Rink amide-AM resin, and a PEG-based Fmoc-Rink amide resin.

A preferred embodiment of the immediately foregoing aspect of the present invention is characterized in that said resin capable of generating a peptide amide is Fmoc-Rink amide-MBHA resin.

A preferred embodiment of the immediately foregoing aspect of the present invention is characterized in that the step (c) comprises the steps of:

(c-1) filtering to remove the resin to yield a (Aib^{8,35})hGLP-1(7-36)-NH₂ (SEQ ID NO:2)/cleavage cocktail filtrate;

(c-2) concentrating the (Aib^{8,35})hGLP-1(7-36)-NH₂ (SEQ ID NO:2)/cleavage cocktail filtrate; (c-3) precipitating crude (Aib^{8,35})hGLP-1(7-36)-NH₂ (SEQ ID NO:2) from the concentrated (Aib^{8,35})hGLP-1(7-36)-NH₂ (SEQ ID NO:2)/cleavage cocktail filtrate;

(c-4) slurrying the crude precipitated (Aib^{8,35})hGLP-1(7-36)-NH₂ (SEQ ID NO:2) in ammonium acetate buffer to perform N—O shift reversal;

(c-5) adjusting the pH of the slurry to yield a solution of (Aib^{8,35})hGLP-1(7-36)-NH₂ (SEQ ID NO:2); and

(c-6) isolating and purifying (Aib^{8,35})hGLP-1(7-36)-NH₂ (SEQ ID NO:2).

A preferred embodiment of the immediately foregoing aspect of the present invention is characterized in that said N—O shift reversal in the step (c-4) is performed by holding the crude precipitated (Aib^{8,35})hGLP-1(7-36)-NH₂ (SEQ ID NO:2) in a slightly basic medium and then bringing the pH back down to about from 3 to 3.7.

A preferred embodiment of the immediately foregoing aspect of the present invention is characterized in that said removal of the Fmoc group from the resin is performed using piperidine in DMF.

A preferred embodiment of the immediately foregoing aspect of the present invention is characterized in that the concentration of said piperidine in DMF is about 25% (v/v).

A preferred embodiment of the immediately foregoing aspect of the present invention is characterized in that the amino acid residues of (Aib^{8,35})hGLP-1(7-36)-NH₂ (SEQ ID NO:2) are coupled using a coupling reagents combination selected from the group consisting of TBTU/HOBt, TBTU/HBTU/DIEA, HATU/DIEA, HCTU/DIEA, TBTU/HOBt/DIEA, DIC/HOBt, DIC/HOAt, HATU/HOBt/DIEA, and HCTU/HOBt/DIEA.

A preferred embodiment of the immediately foregoing aspect of the present invention is characterized in that the amino acid residues of (Aib^{8,35})hGLP-1(7-36)-NH₂ (SEQ ID NO:2) are coupled using a coupling reagents combination of either TBTU/HOBt or TBTU/HBTU/DIEA.

A preferred embodiment of the immediately foregoing aspect of the present invention is characterized in that the amino acid residues of (Aib^{8,35})hGLP-1(7-36)-NH₂ (SEQ ID NO:2) are coupled using a coupling reagents combination of TBTU/HOBt.

A preferred embodiment of the immediately foregoing aspect of the present invention is characterized in that the amino acid residues of (Aib^{8,35})hGLP-1(7-36)-NH₂ (SEQ ID NO:2) are coupled using about 3.0 equivalents of each Fmoc-amino acid, about 2.94 equivalents of TBTU, about 2.94 equivalents of HOBt, and about 4.5 equivalents of DIEA, in about 5 volumetric excesses of DMF.

### DETAILED DESCRIPTION

The application employs the following abbreviations:
ACN acetonitrile
AM aminomethyl
Boc tert-butyloxycarbonyl
DCM dichloromethane
DIC N,N′-diisopropylcarbodiimide
DIEA N,N-diisopropylethylamine
DMF dimethylformamide
DTT dithiothreitol
EDT ethanedithiol
Fmoc Fluorenylmethyloxycarbonyl
HATU O-(7-azabenzotriazol-1-yl)-1,1,3,3-tetramethyluronium hexafluorophosphate
HBTU 2-(1H-benzotriazol-1-yl)-1,1,3,3-tetramethyluronium hexafluorophosphate
HOAt 1-hydroxy-7-azabenzotriazole
HOBt 1-hydroxybenzotriazole
HPLC high pressure liquid chromatography
LOD loss on drying
MBHA 4-methylbenzhydrylamine
MTBE methyl tert-butyl ether
OtBu tert-butyl ester
Pbf 2,2,4,6,7-pentamethyldihydrobenzofuran-5-sulfonyl
PEG polyethylene glycol
TBTU 2-(1H-benzotriazole-1-yl)-1,1,3,3-tetramethyluronium tetrafluoroborate
tBu tert-butyl ether
TES triethylsilane
TFA trifluoroacetic acid
TIPS triisopropylsilane
Trt trityl

A PEG-based Fmoc-Rink amide resin is a resin with an Fmoc-Rink amide linker where the constituent beads of the resin include a PEG component. Some nonexclusive examples of PEG-based Fmoc-Rink amide resins are NovaPeg, NovaGel and AM SURE.

The term “cleavage cocktail” as used herein refer to a mixture of reagents used to remove, or cleave, the assembled peptide from a resin. In addition, a cleavage cocktail also serves to remove all sidechain protecting groups and the N-terminal protecting groups.

The term “about” as used herein in association with parameters or amounts, means that the parameter or amount is within +5% of the stated parameter or amount. The following example is described for purposes of illustrating a method of the present invention and is not to be construed to limit the present invention in any way.

Synthesis of (Aib^{8,35})hGLP-1(7-36)-NH (SEQ ID NO:2)

(Aib^{8,35})hGLP-1(7-36)-NH₂ (SEQ ID NO:2) was synthesized in a 35-liter glass reactor (Quark, Vineland, N.J., USA) equipped with a compressed air motor and PTFE agitator. Fmoc Rink amide MBHA resin (Merck Biosciences, Darmstadt, Germany) with an incorporation of 0.63 mmol/g was used. The Fmoc amino acids (Synthetech Inc., Albany, Oreg., USA) were used with the following side chain protection: Fmoc-Arg(Pbf)-OH, Fmoc-Asp(OtBu)-OH, Fmoc-Glu(OtBu)-OH, Fmoc-Gln(Trt)-OH, Boc-His(Trt)-OH, Fmoc-Lys(Boc)-OH, Fmoc-Ser(tBu)-OH, Fmoc-Thr(tBu)-OH, Fmoc-Trp(Boc)-OH, and Fmoc-Tyr(tBu)-OH. The following Fmoc amino acids did not require side chain protection: Fmoc-Aib-OH, Fmoc-Ala-OH, Fmoc-Gly-OH, Fmoc-Ile-OH, Fmoc-Leu-OH, Fmoc-Phe-OH, and Fmoc-Val-OH.

The synthesis was carried out on a 0.63 mole scale (1 kg input resin). The first 29 amino acids (all except the N-terminal histidine) were coupled using 3.0 equivalents of amino acid and preactivated with 2.94 equivalents of TBTU (Fluka, Seelze, Germany), 2.94 equivalents of HOBt (Fluka, Seelze, Germany), and 4.5 equivalents of DIEA (Sigma-Aldrich, Gillingham, UK) in 4.5 liters of DMF. Coupling times were 60 minutes. Boc-His(Trt)-OH was coupled using 3.4 equivalents of amino acid, 4.08 equivalents of HATU (Applied Biosystems, Framingham, Mass., USA), and 9 equivalents of DIEA in 4.5 liters of DMF. Deprotection of the resin prior to the initial coupling and following each subsequent coupling was performed using 2×10 liters of 25% (v/v) piperidine (BASF, Germany) in DMF.

Upon completion of the peptide assembly on the resin, the resin was washed twice with 10 liters of methanol (Labscan, Dublin, Ireland) and dried to an LOD (loss on drying) of <1% in a vacuum oven (Mason Technology, Dublin, Ireland). The resin was initially dried with nitrogen in the reactor and the final drying took place in the vacuum oven at ambient temperature of approximately 22° C. at <50 mbar. The entire drying process took 3 days. 4200 g of peptidyl-resin was obtained.

The peptide was cleaved from the resin and its sidechain-protecting groups were removed in 6×700 g of sub-lots using a cleavage cocktail of 8.4 liters of TFA/TIPS/water (80/14.3/5.7% v/v) for 170 minutes, for each of the sub-lots. The resin was washed with 0.7 liters of TFA and the filtrates were combined. The cleavage cocktail was concentrated using a rotary evaporator (Buchi, Flawil, Switzerland) to 14-32% its original weight and the crude peptide was precipitated in 13.6-17.5 liters of stirring MTBE (Labscan, Dublin, Ireland). The crude peptide was further washed with 1.5-7.5 liters of MTBE.

Reversal of the N—O shift was performed by slurrying the crude precipitated peptide in ammonium acetate buffer (10 g peptide/100 ml, 10% w/v, i.e., 10 g peptide/100 ml buffer, pH 8-9) for 60 minutes. The pH was brought to 3.3-3.7 with 14-18 liters of glacial acetic acid to give a clear crude peptide solution which had a HPLC purity of about 50%. The peptide solution was filtered through a 0.45-μm filter (Pall Gelman Sciences Inc., New York, N.Y., USA) prior to purification.

The peptide was purified using a reverse-phase preparative HPLC column (Novasep, Pompey, France) packed with C₁₈ stationary phase (EKA Chemicals AB, Bohus, Sweden). Purification was performed under gradient elution using 0.1% TFA in water and acetonitrile.

A salt exchange chromatographic step was carried out using ammonium acetate and acetic acid buffers to generate the acetate salt. Specifically, the peptide was loaded on the HPLC column. The peptide was washed on the column with ammonium acetate buffer for 1 hour, then eluted from the column with an acetic acid/acetonitrile gradient.

The purity of the purified peptide was >99% based on HPLC analysis. Specifically, the peptide solution was concentrated on a rotary evaporator (max temp 40° C.), and the resulting solution was filtered through a 0.45-μm filter (Pall Gelman Sciences Inc., New York, N.Y., USA) and was lyophilized.

The HATU/DIEA system for the final histidine coupling, as compared to the TBTU/HBTU/DIEA, TBTU/HOBt/DIEA, DIC/HOBt, DIC/HOAt, or HATU/HOBt/DIEA system, resulted in better conversion of the 29 mer to (Aib^{8,35})hGLP-1(7-36)-NH₂ (SEQ ID NO:2), and hence increased yield.

In addition, the use of Boc-protected histidine, as compared to Fmoc-protected histidine, gave better yield and allowed for a slight decrease in process time as no Fmoc removal is required prior to cleavage. Statistical design of experimental studies were performed on both the histidine coupling and on cleavage from the resin to select the optimum combination of ratio of reagents and reaction time to increase yield, as shown in the following tables.

As commonly known in the art, N—O shifts are acyl shifts which form in peptides containing threonine or serine residues during exposure to acidic conditions. They result in isomeric impurities which reduce yield and can be difficult to purity. These N—O shifts are reversed by holding the peptide in a slight basic medium (e.g., pH 8-9) and then bringing the pH back down to about 3. The immediately foregoing process allows N—O shift reversal to be performed as a slurry which gives a scale advantage over an entirely solution-based reversal process.

TABLE 1
Small scale design of experiment studies (and yield/purity results
of same) aimed at optimizing the coupling of the N-terminal histidine residue
of (Aib^{8,35})hGLP-1(7-36)-NH₂ (SEQ ID NO: 2)

| Exp | Histidine | HATU/His | DIEA | Reaction | Yield | Purity | D-His | Des-His |
|---|---|---|---|---|---|---|---|---|
| No. | equiv | ratio | equiv | Time | (%) | (%) | Level (%) | Level (%) |
| 1 | 3 | 0.6 | 4.5 | 0.5 | 18.52 | 56.52 | 0.1 | 0.27 |
| 2 | 6 | 0.6 | 4.5 | 4 | 23.51 | 62.76 | 0.14 | 0.06 |
| 3 | 3 | 1.2 | 4.5 | 4 | 26.44 | 63.43 | 0.05 | 0.1 |
| 4 | 6 | 1.2 | 4.5 | 0.5 | 13.17 | 31.94 | 0.07 | 1.29 |
| 5 | 3 | 0.6 | 9 | 4 | 25.18 | 56.47 | 0.14 | 0.16 |
| 6 | 6 | 0.6 | 9 | 0.5 | 21.09 | 53.89 | 0.11 | 0.14 |
| 7 | 3 | 1.2 | 9 | 0.5 | 21.65 | 58.93 | 0.06 | 0.2 |
| 8 | 6 | 1.2 | 9 | 4 | 27.50 | 62.48 | 0.03 | 0.11 |
| 9 | 4.5 | 0.9 | 6.75 | 2.25 | 25.73 | 58.87 | 0.08 | 0.06 |
| 10 | 4.5 | 0.9 | 6.75 | 2.25 | 26.61 | 61.1 | 0.06 | 0.06 |

Note:
Results shown include levels of the impurities related to this coupling (D- and Des-Histidine) in the crude peptide

TABLE 2
Results for repeat small and large scale syntheses using optimized
histidine coupling conditions (3.4 equiv Boc-His, 4.08 equiv
HATU, 9.0 equiv DIEA, and 2.9 hrs reaction time)

| Scale | | | | Des-His Level |
|---|---|---|---|---|
| (input resin) | Yield (%) | Purity (%) | D-His Level (%) | (%) |
| 0.5 g | 29.2 | 57.7 | 2.5 | 1.7 |
| 0.5 g | 30.4 | 59.4 | 2.7 | 3.3 |
| 0.5 g | 27.8 | 56.0 | 3.8 | 2.7 |
| 160 g | 27.8 | 56.01 | 3.19 | 1.93 |
| 160 g | 29.2 | 52.22 | 2.40 | 0.90 |

Note:
Results shown include levels of the impurities related to this coupling (D- and Des-Histidine) in the crude peptide

TABLE 3
Small scale design of experiment studies (and yield/purity
results of same) aimed at optimizing the cleavage of (Aib^{8,35})hGLP-
1(7-36)-NH₂ (SEQ ID NO: 2) from resin

| | Volumetric | | | | |
|---|---|---|---|---|---|
| | excess of cocktail | | TIPS/H₂O | | |
| Exp No. | over resin | Time | ratio | Yield (%) | Purity (%) |
| 1 | 5 | 0.5 | 1 | 3.9 | 12.6 |
| 2 | 15 | 0.5 | 1 | 14.7 | 29.4 |
| 3 | 5 | 4 | 1 | 24.1 | 44.9 |
| 4 | 15 | 4 | 1 | 30.3 | 52.1 |
| 5 | 5 | 0.5 | 4 | 6.9 | 25.6 |
| 6 | 15 | 0.5 | 4 | 20.1 | 47 |
| 7 | 5 | 4 | 4 | 23.9 | 47.9 |
| 8 | 15 | 4 | 4 | 29.6 | 56.6 |
| 9 | 10 | 2.25 | 2.5 | 29.1 | 58.8 |
| 10 | 10 | 2.25 | 2.5 | 29.4 | 58.9 |

Note:
TFA constituted 80% of cleavage cocktail in the above experiments

TABLE 4
Cleavage Optimisation experiments: 12 volumes cleavage
cocktail (over resin weight), 80% TFA, 2.8 hrs reaction
time, and 2.5:1 TIPS:H₂O ratio

| Input resin scale | Yield (%) | Purity (%) |
|---|---|---|
| 10 g | 29.1 | 51.9 |
| 100 g | 27.8 | 51.5 |
| 100 g | 27.1 | 52.7 |

Note:
Results above are post N—O shift reversal

As shown in above Tables 2 and 4, crude work up (incorporating evaporation of the cleavage cocktail and precipitation in MTBE) was optimized to allow precipitation at large scale without impacting on yield. Ultimately, a large-scale synthesis (1 kg input resin) was performed which was subsequently cleaved in sub-lots with an overall synthetic yield of 27%, which represents approximately 8% increase compared with previous methods' yields at lower scales.

For purification method development, efforts were focused on modifying the TFA gradient used from the outset to minimize the number of purification passes required to obtain material at >99% purity, which resulted in purification yields of 50-60%.

### Other Embodiments

From the above description, one skilled in the art can easily ascertain the essential characteristics of the present invention, and without departing from the spirit and scope thereof, can make various changes and modifications of the invention to adapt it to various uses and conditions. Thus, other embodiments are also within the claims.

1. A process for the synthesis of (Aib^{8,35})hGLP-1(7-36)-NH₂ (SEQ ID NO:2), which comprises stepwise solid-phase Fmoc-chemistry.

2. A process for the synthesis of (Aib^{8,35})hGLP-1(7-36)-NH₂ (SEQ ID NO:2) according to claim 1, comprising the steps of:
(a) successively coupling Fmoc-amino acids, from the C-terminus to the N-terminus of (Aib^{8,35})hGLP-1(8-35)-NH₂ (SEQ ID NO:8), with a sidechain-protected Arg resin, wherein the Fmoc group is removed from the N-terminus after each successive coupling step, to yield a sidechain-protected Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:4);
(b) coupling sidechain-protected Boc-His-OH with the sidechain-protected Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:4) to yield a sidechain-protected Boc-His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:5);
(c) treating the sidechain-protected Boc-His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:5) with a cleavage cocktail and removing the sidechain-protecting groups and the N-terminus protecting group therefrom to yield crude (Aib^{8,35})hGLP-1(7-36)-NH₂ (SEQ ID NO:2); and
(d) isolating and purifying the crude (Aib^{8,35})hGLP-1(7-36)-NH₂ (SEQ ID NO:2) to yield purified (Aib^{8,35})hGLP-1(7-36)-NH₂ (SEQ ID NO:2).

3. A process for the synthesis of (Aib^{8,35})hGLP-1(7-36)-NH₂ (SEQ ID NO:2) according to claim 2, further comprising the steps of:
(a-1) deprotecting an Fmoc-protected resin capable of generating a peptide amide to remove the Fmoc group from the resin;
(a-2) attaching sidechain-protected Fmoc-Arg-OH onto the resin to yield a sidechain-protected Fmoc-Arg resin; and
(a-3) removing the Fmoc group from the sidechain-protected Fmoc-Arg resin to yield a sidechain-protected Arg resin;
which precede the step (a).

4. A process for the synthesis of (Aib^{8,35})hGLP-1(7-36)-NH₂ (SEQ ID NO:2) according to claim 3, wherein:
said sidechain-protected Fmoc-Arg-OH in the step (a-2) is Fmoc-Arg(Pbf)-OH;
said sidechain-protected Fmoc-Arg resin is Fmoc-Arg(Pbf) resin;
said sidechain-protected Arg resin is sidechain-protected Arg(Pbf) resin;
said Fmoc-amino acids from the C-terminus to the N-terminus of the formula (Aib^{8,35})hGLP-1(8-35)-NH₂ (SEQ ID NO:8) are Fmoc-Aib-OH, Fmoc-Lys(Boc)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(Boc)-OH, Fmoc-Ala-OH, Fmoc-Ala-OH, Fmoc-Gln(Trt)-OH, Fmoc-Gly-OH, Fmoc-Glu(OtBu)-OH, Fmoc-Leu-OH, Fmoc-Tyr(tBu)-OH, Fmoc-Ser(tBu)-OH, Fmoc-Ser(tBu)-OH, Fmoc-Val-OH, Fmoc-Asp(OtBu)-OH, Fmoc-Ser(tBu)-OH, Fmoc-Thr(tBu)-OH, Fmoc-Phe-OH, Fmoc-Thr(tBu)-OH, Fmoc-Gly-OH, Fmoc-Glu(OtBu)-OH, and Fmoc-Aib-OH;
said sidechain-protected Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:4) is Aib-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Aib-Arg(Pbf) resin (SEQ ID NO:6);
said sidechain-protected Boc-His-OH is Boc-His(Trt)-OH;
said sidechain-protected Boc-His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:5) is Boc-His(Trt)-Aib-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Aib-Arg(Pbf) resin (SEQ ID NO:7); and
said cleavage cocktail is selected from the group consisting of TFA/TIPS/water cleavage cocktail, TFA/TIPS/DCM cleavage cocktail, TFA/phenol/water/TIPS cleavage cocktail, TFA/phenol/water/thioanisole/EDT cleavage cocktail, TFA/phenol/water/thioanisole/1-dodecanethiol cleavage cocktail, TFA/DTT/water/TIPS cleavage cocktail, TFA/phenol cleavage cocktail, TFA/phenol/methanesulfonic acid cleavage cocktail, TFA/thioanisole/EDT/anisole cleavage cocktail, TFA/TES cleavage cocktail, TFA/water cleavage cocktail, TFA/DCM/indole cleavage cocktail, and TFA/TIPS cleavage cocktail.

5. A process for the synthesis of (Aib^{8,35})hGLP-1(7-36)-NH₂ (SEQ ID NO:2) according to claim 4, wherein said resin capable of generating a peptide is selected from the group consisting of Fmoc-Rink amide-MBHA resin, Fmoc-Rink amide-AM resin, a PEG-based Fmoc-Rink amide resin, and Sieber amide resin.

6-7. (canceled)

8. A process for the synthesis of (Aib^{8,35})hGLP-1(7-36)-NH₂ (SEQ ID NO:2) according to claim 5, wherein the step (d) comprises the steps of:
(d-1) filtering to remove the resin to yield a (Aib^{8,35})hGLP-1(7-36)-NH₂ (SEQ ID NO:2)/cleavage cocktail filtrate;
(d-2) concentrating the (Aib^{8,35})hGLP-1(7-36)-NH₂ (SEQ ID NO:2)/cleavage cocktail filtrate;
(d-3) precipitating crude (Aib^{8,35})hGLP-1(7-36)-NH₂ (SEQ ID NO:2) from the concentrated (Aib^{8,35})hGLP-1(7-36)-NH₂ (SEQ ID NO:2)/cleavage cocktail filtrate;
(d-4) slurrying the crude precipitated (Aib^{8,35})hGLP-1(7-36)-NH₂ (SEQ ID NO:2) in ammonium acetate buffer to perform N—O shift reversal;
(d-5) adjusting the pH of the slurry to yield a solution of (Aib^{8,35})hGLP-1(7-36)-NH₂ (SEQ ID NO:2); and
(d-6) isolating and purifying (Aib^{8,35})hGLP-1(7-36)-NH₂ (SEQ ID NO:2).

9. The process for the synthesis of (Aib^{8,35})hGLP-1(7-36)-NH₂ (SEQ ID NO:2) according to claim 8, wherein said N—O shift reversal is performed by holding the crude precipitated (Aib^{8,35})hGLP-1(7-36)-NH₂ (SEQ ID NO:2) in a slightly basic medium and then bringing the pH back down to about from 3 to 3.7.

10. The process for the synthesis of (Aib^{8,35})hGLP-1(7-36)-NH₂ (SEQ ID NO:2) according to claim 9, wherein said removal of the Fmoc group from the resin is performed using piperidine in DMF.

11. The process for the synthesis of (Aib^{8,35})hGLP-1(7-36)-NH₂ (SEQ ID NO:2) according to claim 10, wherein the concentration of said piperidine in DMF is about 25% (v/v).

12. The process for the synthesis of (Aib^{8,35})hGLP-1(7-36)-NH₂ (SEQ ID NO:2) according to claim 2, wherein the amino acid residues of (Aib^{8,35})hGLP-1(7-36)-NH₂ (SEQ ID NO:2) are coupled using a coupling reagents combination selected from the group consisting of TBTU/HOBt, TBTU/HBTU/DIEA, HATU/DIEA, HCTU/DIEA, TBTU/HOBt/DIEA, DIC/HOBt, DIC/HOAt, HATU/HOBt/DIEA, and HCTU/HOBt/DIEA.

13. The process for the synthesis of (Aib^{8,35})hGLP-1(7-36)-NH₂ (SEQ ID NO:2) according to claim 12, wherein:
the first 29 amino acid residues of (Aib^{8,35})hGLP-1(7-36)-NH₂ (SEQ ID NO:2) from the C-terminus are coupled using a coupling reagents combination of either TBTU/HOBt or TBTU/HBTU/DIEA; and
the N-terminal histidine is coupled using a coupling reagents combination selected from the group consisting of HATU/DIEA, HCTU/DIEA, TBTU/HBTU/DIEA, TBTU/HOBt/DIEA, DIC/HOBt, DIC/HOAt, HATU/HOBt/DIEA, and HCTU/HOBt/DIEA.

14. The process for the synthesis of (Aib^{8,35})hGLP-1(7-36)-NH₂ (SEQ ID NO:2) according to claim 13, wherein:
said coupling reagents combination used for coupling the first 29 amino acid residues of (Aib^{8,35})hGLP-1(7-36)-NH₂ (SEQ ID NO:2) from the C-terminus is TBTU/HOBt; and
said coupling reagents combination used for coupling the N-terminal histidine is HATU/DIEA.

15. The process for the synthesis of (Aib^{8,35})hGLP-1(7-36)-NH₂ (SEQ ID NO:2) according to claim 14, wherein:
the first 29 amino acid residues of (Aib^{8,35})hGLP-1(7-36)-NH₂ (SEQ ID NO:2) from the C-terminus are coupled using about 3.0 equivalents of each Fmoc-amino acid, about 2.94 equivalents of TBTU, about 2.94 equivalents of HOBt, and about 4.5 equivalents of DIEA, in about 5 volumetric excesses of DMF; and
the N-terminal histidine is coupled using about 3.4 equivalents of Boc-His(Trt)-OH, about 4.08 equivalents of HATU, and about 9.0 equivalents of DIEA, in about 5 volumetric excesses of DMF.

16. A process for the synthesis of (Aib^{8,35})hGLP-1(7-36)-NH₂ (SEQ ID NO:2) according to claim 1, comprising the steps of:
(a) successively coupling Fmoc-amino acids, from the C-terminus to the N-terminus of (Aib^{8,35})hGLP-1(7-35)-NH₂ (SEQ ID NO:9), with a sidechain-protected Arg resin, wherein the Fmoc group is removed from the N-terminus after each successive coupling step, to yield a sidechain-protected His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:3);
(b) treating the sidechain-protected His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:3) with a cleavage cocktail and removing sidechain-protecting groups therefrom to yield crude (Aib^{8,35})hGLP-1(7-36)-NH₂ (SEQ ID NO:2); and
(c) isolating and purifying the crude (Aib^{8,35})hGLP-1(7-36)-NH₂ (SEQ ID NO:2) to yield purified (Aib^{8,35})hGLP-1(7-36)-NH₂ (SEQ ID NO:2).

17. A process for the synthesis of (Aib^{8,35})hGLP-1(7-36)-NH₂ (SEQ ID NO:2) according to claim 16, further comprising the steps of:
(a-1) deprotecting an Fmoc-protected resin capable of generating a peptide amide to remove the Fmoc group from the resin;
(a-2) attaching sidechain-protected Fmoc-Arg-OH onto the resin to yield a sidechain-protected Fmoc-Arg resin; and
(a-3) removing the Fmoc group from the sidechain-protected Fmoc-Arg resin to yield a sidechain-protected Arg resin;
which precede the step (a).

18. A process for the synthesis of (Aib^{8,35})hGLP-1(7-36)-NH₂ (SEQ ID NO:2) according to claim 17, wherein:
said sidechain-protected Fmoc-Arg-OH in the step (a-2) is Fmoc-Arg(Pbf)-OH;
said sidechain-protected Fmoc-Arg resin is Fmoc-Arg(Pbf)-OH and Fmoc-Arg(Pbf) resin;
said sidechain-protected Arg resin is sidechain-protected Arg(Pbf) resin;
said Fmoc-amino acids from the C-terminus to the N-terminus of the formula (Aib^{8,35})hGLP-1(7-35)-NH₂ (SEQ ID NO:9) are Fmoc-Aib-OH, Fmoc-Lys(Boc)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(Boc)-OH, Fmoc-Ala-OH, Fmoc-Ala-OH, Fmoc-Gln(Trt)-OH, Fmoc-Gly-OH, Fmoc-Glu(OtBu)-OH, Fmoc-Leu-OH, Fmoc-Tyr(tBu)-OH, Fmoc-Ser(tBu)-OH, Fmoc-Ser(tBu)-OH, Fmoc-Val-OH, Fmoc-Asp(OtBu)-OH, Fmoc-Ser(tBu)-OH, Fmoc-Thr(tBu)-OH, Fmoc-Phe-OH, Fmoc-Thr(tBu)-OH, Fmoc-Gly-OH, Fmoc-Glu(OtBu)-OH, Fmoc-Aib-OH, and Fmoc-His(Trt)-OH;
said sidechain-protected His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg resin (SEQ ID NO:3) is His(Trt)-Aib-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Aib-Arg(Pbf) resin (SEQ ID NO:10); and
said cleavage cocktail is selected from the group consisting of TFA/TIPS/water cleavage cocktail, TFA/TIPS/DCM cleavage cocktail, TFA/phenol/water/TIPS cleavage cocktail, TFA/phenol/water/thioanisole/EDT cleavage cocktail, TFA/phenol/water/thioanisole/1-dodecanethiol cleavage cocktail, TFA/DTT/water/TIPS cleavage cocktail, TFA/phenol cleavage cocktail, TFA/phenol/methanesulfonic acid cleavage cocktail, TFA/thioanisole/EDT/anisole cleavage cocktail, TFA/TES cleavage cocktail, TFA/water cleavage cocktail, TFA/DCM/indole cleavage cocktail, and TFA/TIPS cleavage cocktail.

19. A process for the synthesis of (Aib^{8,35})hGLP-1(7-36)-NH₂ (SEQ ID NO:2) according to claim 18, wherein said resin capable of generating a peptide is selected from the group consisting of Fmoc-Rink amide-MBHA resin, Fmoc-Rink amide-AM resin, a PEG-based Fmoc-Rink amide resin, and Sieber amide resin.

20-21. (canceled)

22. A process for the synthesis of (Aib^{8,35})hGLP-1(7-36)-NH₂ (SEQ ID NO:2) according to claim 19, wherein the step (c) comprises the steps of:
(c-1) filtering to remove the resin to yield a (Aib^{8,35})hGLP-1(7-36)-NH₂ (SEQ ID NO:2)/cleavage cocktail filtrate;
(c-2) concentrating the (Aib^{8,35})hGLP-1(7-36)-NH₂ (SEQ ID NO:2)/cleavage cocktail filtrate;
(c-3) precipitating crude (Aib^{8,35})hGLP-1(7-36)-NH₂ (SEQ ID NO:2) from the concentrated (Aib^{8,35})hGLP-1(7-36)-NH₂ (SEQ ID NO:2)/cleavage cocktail filtrate;
(c-4) slurrying the crude precipitated (Aib^{8,35})hGLP-1(7-36)-NH₂ (SEQ ID NO:2) in ammonium acetate buffer to perform N—O shift reversal;
(c-5) adjusting the pH of the slurry to yield a solution of (Aib^{8,35})hGLP-1(7-36)-NH₂ (SEQ ID NO:2); and
(c-6) isolating and purifying (Aib^{8,35})hGLP-1(7-36)-NH₂ (SEQ ID NO:2).

23. The process for the synthesis of (Aib^{8,35})hGLP-1(7-36)-NH₂ (SEQ ID NO:2) according to claim 22, wherein said N—O shift reversal in the step (c-4) is performed by holding the crude precipitated (Aib^{8,35})hGLP-1(7-36)-NH₂ (SEQ ID NO:2) in a slightly basic medium and then bringing the pH back down to about from 3 to 3.7.

24. The process for the synthesis of (Aib^{8,35})hGLP-1(7-36)-NH₂ (SEQ ID NO:2) according to claim 23, wherein said removal of the Fmoc group from the resin is performed using piperidine in DMF.

25. The process for the synthesis of (Aib^{8,35})hGLP-1(7-36)-NH₂ (SEQ ID NO:2) according to claim 24, wherein the concentration of said piperidine in DMF is about 25% (v/v).

26. The process for the synthesis of (Aib^{8,35})hGLP-1(7-36)-NH₂ (SEQ ID NO:2) according to claim 16, wherein the amino acid residues of (Aib^{8,35})hGLP-1(7-36)-NH₂ (SEQ ID NO:2) are coupled using a coupling reagents combination selected from the group consisting of TBTU/HOBt, TBTU/HBTU/DIEA, HATU/DIEA, HCTU/DIEA, TBTU/HOBt/DIEA, DIC/HOBt, DIC/HOAt, HATU/HOBt/DIEA, and HCTU/HOBt/DIEA.

27-28. (canceled)

29. The process for the synthesis of (Aib^{8,35})hGLP-1(7-36)-NH₂ (SEQ ID NO:2) according to claim 26, wherein the amino acid residues of (Aib^{8,35})hGLP-1(7-36)-NH₂ (SEQ ID NO:2) are coupled using about 3.0 equivalents of each Fmoc-amino acid, about 2.94 equivalents of TBTU, about 2.94 equivalents of HOBt, and about 4.5 equivalents of DIEA, in about 5 volumetric excesses of DMF.
